(19) United States
(12) Patent Application Publication
(10) Pub. No.: US 2005/0194400 A1
(43) Pub. Date: Sep. 8, 2005

(54) Children's nasal spray containers and inhalers

(76) Inventors: Patricia J. Berube, Cohasset, MA (US); Amy L. White, Hanover, MA (US); Jennifer Brooks Raymond, Cohasset, MA (US)

Correspondence Address: MILDE & HOFFBERG, LLP, 10 BANK STREET, SUITE 460, WHITE PLAINS, NY, 10606, US

(21) Appl. No.: 11/051,900

(22) Filed: Feb. 4, 2005

Related U.S. Application Data
(63) Continuation-in-part of application No. 10/892,585, filed on Jul. 16, 2004.
(60) Provisional application No. 60/550,397, filed on Mar. 5, 2004.
(60) Provisional application No. 60/633,642, filed on Dec. 6, 2004.

(51) Int. Cl. A61M 11/00

(52) U.S. Cl. 222/78; 222/175

(57) ABSTRACT

A container for dispensing nasal spray, ear drops, or an inhalant mist, formed in the three-dimensional shape of an entertaining figurine, such as an elephant, or a panda bear, having appendages which serve as handles for grasping and manipulating the container to administer the spray or mist to a child. The container for dispensing an inhalant mist preferably has a mechanism for counting the number of puffs dispensed and for displaying the count.

### CROSS-REFERENCE TO RELATED APPLICATIONS

This application is a continuation-in-part of U.S. Provisional Patent Application Ser. No. 60/550,397, filed Mar. 5, 2004; U.S. patent application Ser. No. 10/892,585, filed Jul. 16, 2004, and U.S. Provisional Patent Application Ser. No. 60/633,642, filed Dec. 6, 2004, entitled “CHILDREN'S NASAL SPRAY CONTAINERS AND INHALERS”.

### FIELD OF THE INVENTION

The present invention relates to containers for dispensing nasal sprays, ear drops and nasal mist inhalant, and specifically to containers which are “user-friendly” for children.

### BACKGROUND OF THE INVENTION

Common dispensing containers for nasal sprays include the squeeze bottle containers, and the pump action containers. Examples of the squeeze bottle nasal spray containers may be seen in U.S. Pat. No. 4,513,891 and also the references cited therein. Typically, these containers comprise a nozzle fitted to the top of a container having flexible side walls, and containing a reservoir of fluid to be dispensed, and means, such as a dip tube for conveying the fluid to the nozzle when the side walls of the container are squeezed.

During dispensing of the spray, the nozzle is disposed directly under, or within, the nostril while the container is squeezed to dispense the fluid spray into the nostril. The precise positioning, and coordinated movement required to dispense the spray is considered too complicated to be performed by a child. While it may be easy for an adult to use these dispensers on themselves, it is much more difficult for an adult to administer the spray to a child.

A pump action container typically includes a hard walled container, and a horizontally extending finger platform at the bottom of the nozzle. The throat of the container is disposed up into the nozzle, such as by placing the opposed thumb on the bottom of the container and pushing towards the fingers, to dispense a fluid spray from the nozzle. A spring action returns the container to its original position, with the throat beneath the finger platform, ready for its next use.

Through the years each type of container has been improved to produce uniform, reproducible, fine sprays of fluid. However, the precise positioning and coordinated movement required to use these devices has not changed. In addition, little attempt has been need to design a container which is “user-friendly” for children.

Similarly, inhalers have been developed to carefully, and reproducibly produce a fine mist of fluid droplets or microdust to be inhaled through the mouth into the bronchial tubes. Typically, the inhaler includes a pump action container which dispenses the spray into a breathing tube, at right angles to the pumping action. The other end of the breathing tube is placed within the mouth prior to actuating the pump. Using the fingers and opposed thumb to pump the container, the fine mist is released into the breathing tube from a medication canister, to be instantly inhaled by the patient. As with the nasal spray containers, careful placement and coordinated movement is required to use these devices. And again, little attempt has been made to design an inhaler which is “user-friendly” for children.

Inhalant dispensers of this type, called “metered-dosed inhalers” can dispense asthma medication directly to the lungs to prevent and treat asthma attacks, and/or can disperse diabetes medication in measured doses.

Medication canisters, used with these devices, may dispense asthma medications, which include but are not limited to, albuterol, USP, proventil, ventolin, fluticasone propionate (hereinafter referred to as “asthma medication”). A typical canister, sold by prescription only, may be obtained from the following pharmaceutical companies, which include but are not limited to, Warrick Pharmaceuticals Corporation, Shering/Keys Pharmaceutical, GlaxoSmithKline (hereinafter referred to as “Pharmaceutical Companies”). Each of the Pharmaceutical Companies asthma medication notes the number of “puffs” per canister. Such canisters deliver an average of 200 metered inhalations of microcrystalline suspension of asthma medication in a propellant, such as dichlorodifluoromethane with oleic acid, per canister. Even after all of the active asthma medication has been dispensed, there is sufficient propellant left in the canister to provide 50-100 more further audible “puffs” beyond average metered dose.

If patients, particularly children, are not taught to recognize when an inhaler is empty, their lives are at risk if there is no medication or if they continue to use the medication canister past its intended and medically effective, duration.

Medication canisters, used with inhalant dispensers, may also dispense diabetes medications. Two such medications which have been approved by the U.S. Federal Drug Administration (FDA) are:
(1) Brand Name: EXUBERA
   Source: Aventis and Pfizer
(2) Brand Name: AERx Diabetes
   Source: Novo Nordisk and Aradigm

Hence, there is a need for an inhalant dispenser which counts the number of doses used from the total available number of doses listed by the manufacturer. More particularly, there is a need to provide a reliable way of counting the puffs dispensed from a “metered-dose inhaler”.

### SUMMARY OF THE INVENTION

It is an object of the present invention to provide a container for dispensing a nasal spray, ear drops or an inhalant mist, which is easy to use with a child.

It is a further object of the present invention to provide a nasal spray container, or an inhaler, which decreases the fear of the child, and engages the interest of the child. It is a further object of the invention to provide a nasal spray container, or and inhaler, which elicits the participation of the child in receiving the spray or mist.

It is a still further object of the present invention to provide a “user friendly” inhaler for children which counts the number of puffs dispensed of asthma medication and displays this count.

These objects, as well as other objects which will become apparent from the discussion that follows, are achieved, in accordance with the present invention which comprises a container for dispensing nasal spray, ear drops or bronchial mist, which container comprises an entertaining, three dimensional figurine of an animal or a toy object, having multiple handles formed of, e.g., the appendages of the animals, and foci of tactile and visual interest, and perhaps aural interest, to distract a child during administration of the mist, and encourage their participation in receiving the spray or mist.

The present invention thus comprises a nasal spray dispensing container, nasal inhaler, or ear drop dispenser fashioned to assist an adult in administering the nasal spray, bronchial mist or ear drops to a child by configuring the container as an entertaining three-dimensional figurine, or toy. The containers of the present invention distract a child from the task of administering a fluid, such as a pharmaceutical preparation, by providing an alternative focus; items of tactile and visual interest; and a reminder of a toy, or a play stimulus.

At the same time, the container is provided with multiple handles. These handles enable the adult to administer the spray. mist or drops to a child, and assist an older child learn to use such a dispensing container. When an adult administers the spray, mist or drops to a child, the nostril, mouth or ear may be a moving target. Thus the task of positioning the spray of mist container, and the coordinated movement to dispense the fluid are vastly complicated. In addition, though an adult using such a dispenser on themselves positions and squeezes/pumps in front of their own face, when administering to a child, both the positioning and the coordinated movement to dispense may have to be performed at awkward angles and positions. The many handles on the containers of the present invention aid the adult in maintaining a grasp on the container, and moving it within their fingers, to be able to perform the coordinated movement to dispense the spray or mist.

Likewise, when administering ear drops to a child, any means, such as watching or holding the ear drop container, may keep their ears from moving, and thereby assist the adult in administering the drops. In addition, when the ear drop container is in the shape of an animal figurine, making the head of the figurine into a side-hinged, “flip-top”, which exposes the dropper, also places the head of the figurine in a mimicking, “ear-up” position, required to dispense ear drops. Then, if the child mimic the head position of the figurine, it will be easier to administer the drops. Similarly, the front-hinged head of the figurine on a nasal spray container could place the nostrils of the animal down, the position needed to properly administer nasal spray.

The three-dimensional figurine may be formed about, configured into, or configured to contain replaceable units of, a squeeze bottle nasal spray dispensing container, a pump action nasal spray dispensing container, a pump action inhaler, an ear drop container with dispenser, or any future construction or spray or mist dispenser. The appendages of the figurine, such as the legs, ears, tail, wings, etc. of an animal figure, may provide additional handles, or means for grasping, holding, and manipulating the container. In addition, the figurine of the container may be provided with fine fingering details, to interest the small fingers of a child. Graphic details of the figurine, such as its eyes, spots, and its overall coloring may serve as means of visual interest for the child. The three dimensional figurine may be an animal, such as an elephant, giraffe, or bear, or a fantasy figure, such as a unicorn or a dragon.

If desired, a sounding device may be incorporated into the dispensing containers, such that dispensing the spray or mist is preceded by a sound, such as an elephant trumpeting. When using such a container, the child may be encouraged to inhale when they hear the sound.

According to a further feature of the present invention, an inhalant dispenser take the shape of an animal figurine, such as a panda bear, having a torso or body; a head which is removable for the insertion of a canister of inhalant—for example, asthma medication—; and feet which are removable to provide an opening for dispensing the inhalant—for example, to the user's mouth. With this arrangement, a puff of inhalant is released from the canister upon pressing the head downward toward the torso.

According to a still further feature of the present invention, the inhalant dispenser includes a counter mechanism for counting the number of times that the head is pressed downward, thereby counting the number of puffs produced by the canister of inhalant. The counter mechanism preferably displays the count by moving a physical element, such as an elongate strip, cylinder or the like, within the torso of the figurine, such that one of a plurality of numbers imprinted on this element is moved in front of a window opening in the torso. In this way, the user of the inhaler may see either the total number of puffs dispensed or, preferably, the total number of puffs which remain in the inhaler to be dispensed. In the former case, the counter counts up from zero whereas, in the latter case, the counter counts down from the maximum number of puffs—e.g., 200—toward zero.

For a full understanding of the present invention, reference should now be made to the following detailed description of the preferred embodiments of the invention as illustrated in the accompanying drawings.

### BRIEF DESCRIPTION OF THE DRAWINGS

FIG. 1 is a side view of a nasal spray dispensing container according to the present invention, in the shape of an elephant.

FIG. 2 is a side view of unicorn shaped nasal spray dispensing container according to the present invention.

FIG. 2A is a side view of a unicorn dispensing ear drop container, wherein the ear drop containing vessel is encased in a unicorn figurine, with the head and unicorn forming the dropper top.

FIG. 3 is a side view of a giraffe shaped nasal spray dispensing container according to the present invention.

FIG. 3A is a side view of giraffe container for dispensing nasal spray, where the head of the giraffe forms a “flip-top”, hinged at the front, such that when the top is flipped, the dispensing opening is exposed for use, and the nostrils of the giraffe are facing down.

FIG. 4 is a side view of a dragon shaped nasal spray dispensing container according to the present invention.

FIG. 5 is a side view of an inhalant mist dispensing container according to the present invention, shaped like a panda bear.

FIG. 5A is a side view of another embodiment of an inhalant mist dispensing container, wherein the head of the figurine is a “flip-top”, which, when opened, exposes the plunger top of an inhalant dispensing canister.

FIG. 6 is a front view of another embodiment of an ear drop dispensing container wherein the head of the figurine is a “flip-top” hinged at the side, such that the head is turned on its side, placing the ear of the figurine up, and exposing the dropper top for dispensing the ear drops contained therein.

FIGS. 7A, 7B and 7C are front, side and top views, respectively of an inhaler in the shape of a panda bear.

FIG. 8 shows a panda bear inhaler, in front and rear perspective views, respectively, according to the preferred embodiment and the best mode for practicing the present invention.

FIG. 9 is a perspective view of the panda bear inhaler of FIG. 8, as it is being used by a child.

FIG. 10 is a perspective view of the panda bear inhaler of FIGS. 7-9, in the assembled condition.

FIG. 11 is a perspective view of the panda bear inhaler of FIG. 10, in the disassembled condition.

FIG. 12 is a front view of the panda bear inhaler of FIG. 11, showing the puffer counter in the panda's belly.

FIGS. 13A and 13B are side and top views, respectively, of the panda bear inhaler of FIGS. 8-12 with a mechanism for counting puffs by incrementing an elongate puffer counter tape with each puff so that the number appearing in the panda's belly counts down from 200 to 0.

FIGS. 14A and 14B are side and top views, respectively, of the puffer counter tape with a plurality of numbers imprinted thereon for displaying the number of puffs in a window opening in the panda bear torso.

FIG. 15A is a cross-sectional and cutaway view of the panda bear inhaler of FIGS. 8-12, with a mechanism for counting puffs by ratcheting a rotatable cylinder about its axis with each puff so that the number appearing in the window opening in the panda's belly counts up or down.

FIG. 15B is a perspective view of the counting cylinder shown in FIG. 15A, and the mechanism for shifting the counting cylinder in its axial direction to align different rows of numbers with the window opening for display.

### DESCRIPTION OF THE PREFERRED EMBODIMENTS

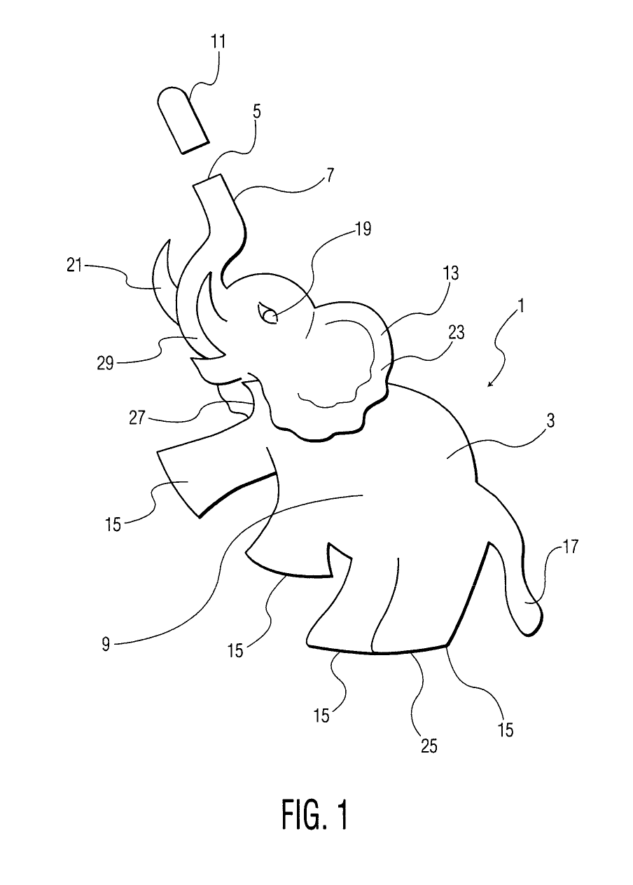

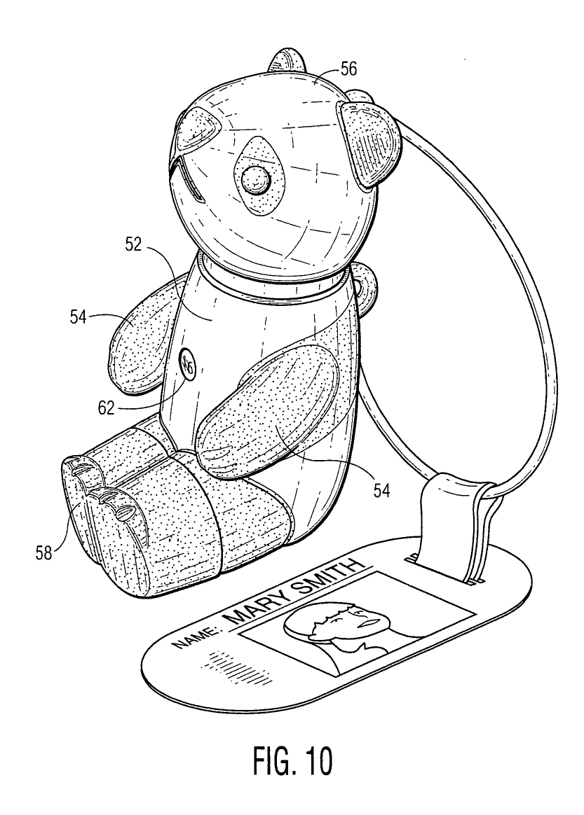

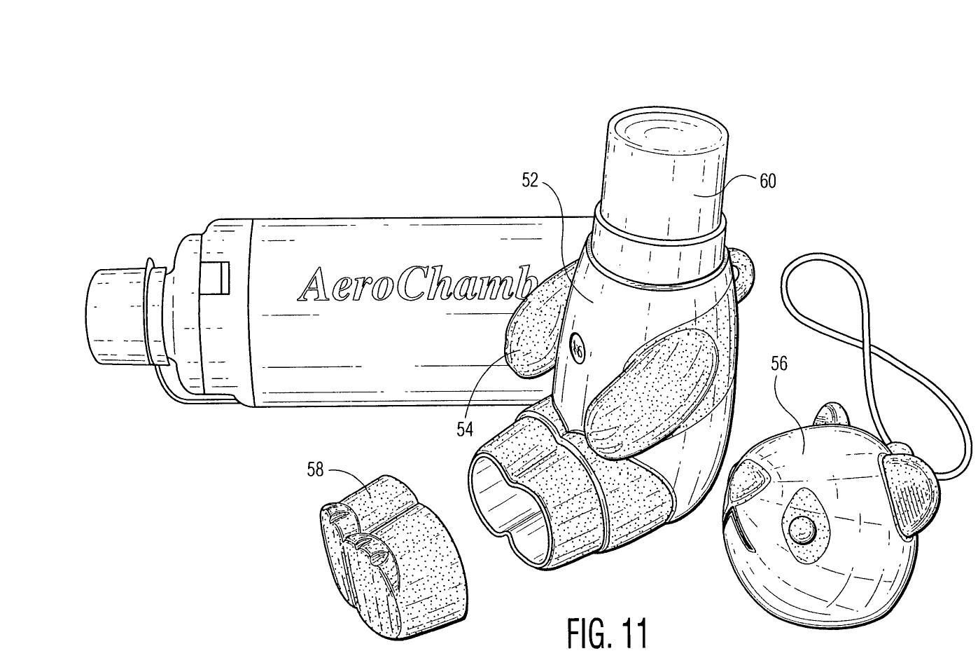

The preferred embodiments of the present invention will now be described with reference to FIGS. 1-15 of the drawings. Identical elements in the various figures are designated with the same reference numerals.

FIG. 1 illustrates a container for dispensing nasal spray, shown generally at 1. The container is formed in the shape of three-dimensional figure, 3, in this embodiment, an elephant. In this particular embodiment, the container is formed such that the sprayed dispensing opening, or nozzle, 5, is formed from the trunk of the elephant. The end of the trunk comprises the insertion region, 7, which may be disposed within the nostril of the child before dispensing the spray. When constructed as a squeeze bottle dispensing container, the container may be squeezed in the activation region, 9, roughly positioned at the center of the flexible side walls of the container. If desired, a sanitary cap, 11, may be provided for the dispensing opening, or nozzle, 5, and the insertion region, 7. The appendages of the elephant, in this embodiment the ears, 13, feet, 15, and tail, 17, provide additional grasping handles for the person administering the nasal spray from the container. They may also provide in regions of fingering interest for the child to whom the nasal spray is being administered. Other finer details of the elephant figurine, such as a tusk, 21, provide further regions of fingering interest for the small exploring fingers of a child. Graphic detail, such as the eyes, 19, provides visual interest for the child.

Alternatively, the elephant figurine container may be configured as a pump action nasal spray dispenser. When so configured, the ears may be extended away from the body of the elephant, to form a finger platform, 23. The hind feet may comprise the thumb placement surface, 25. Displacement of the thumb towards the finger platform may telescope, e.g., the throat, 27, into the head, 29, to dispense the nasal spray from the opening, 5.

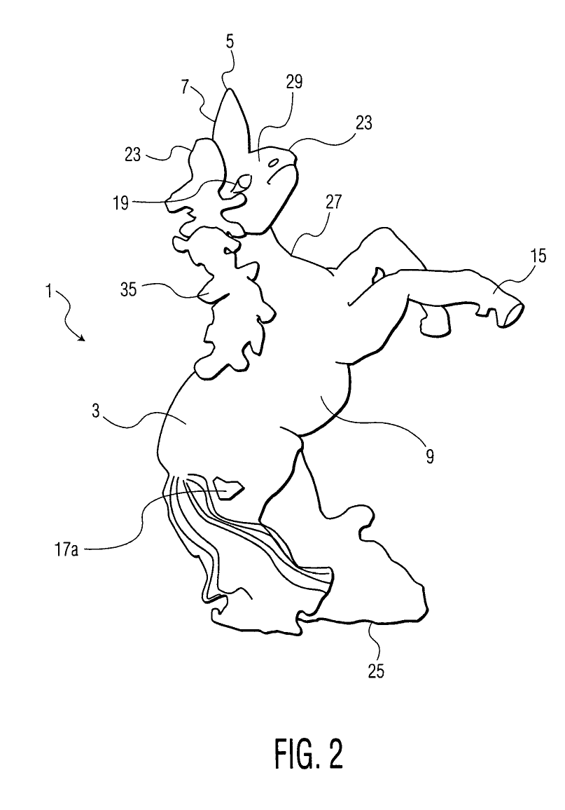

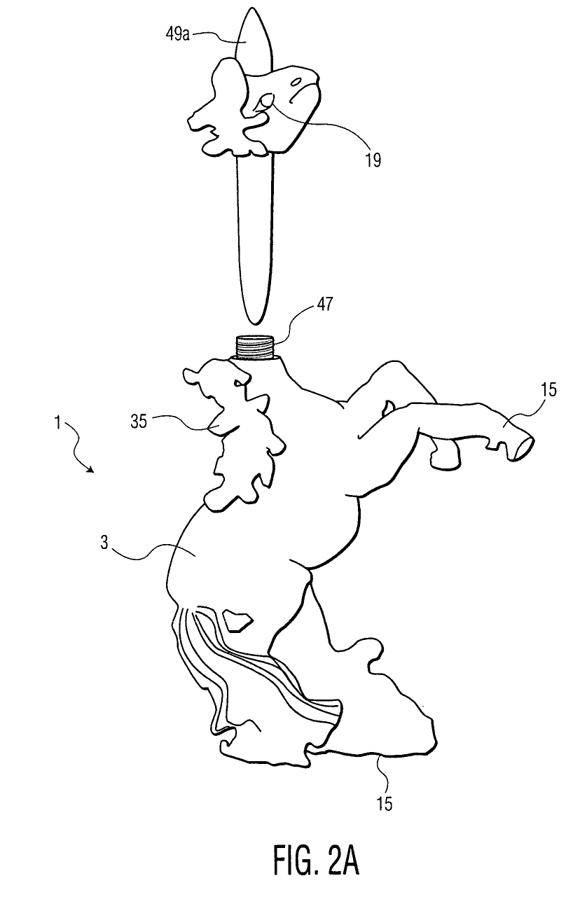

FIG. 2 illustrates an alternative embodiment of nasal spray dispensing container of the present invention, 1, formed in the three-dimensional shape, 3, of a unicorn. In this embodiment that dispensing opening, 5, is formed at the tip of the horn, with the insertion region, 7, comprising the end of the horn, as shown. In the embodiment shown, the mane, 35, forms a fine fingering detail for the child, as does the tail, 17a. When configured as a pump action dispenser, the snout, 29, and the top of the mane, may form the finger platform, 23, and hind feet form the thumb placement region, 25.

FIG. 2A depicts a dropper-dispensing container, 1, for a medicament, such as ear drops. A vessel, such as a glass bottle is encased in the entertaining figurine, 3, which has appendages for grasping the container, fine finger detail, and graphic detail, as described above. In this particular embodiment, the head of the figurine contains a dropper cap, 49a, for dispensing e.g., ear drops. As shown in FIG. 2A, when the dropper cap is unscrewed from the neck of the bottle, 47, the dropper cap may be used to dispense the ear drops.

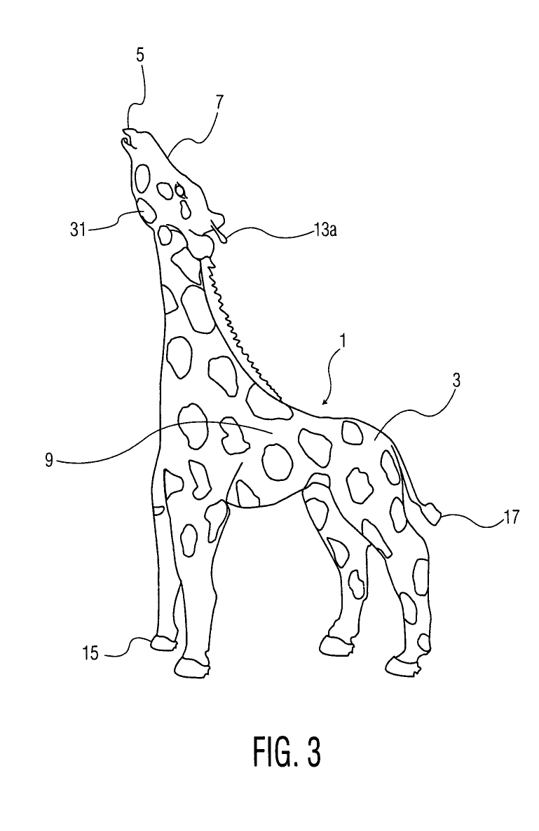

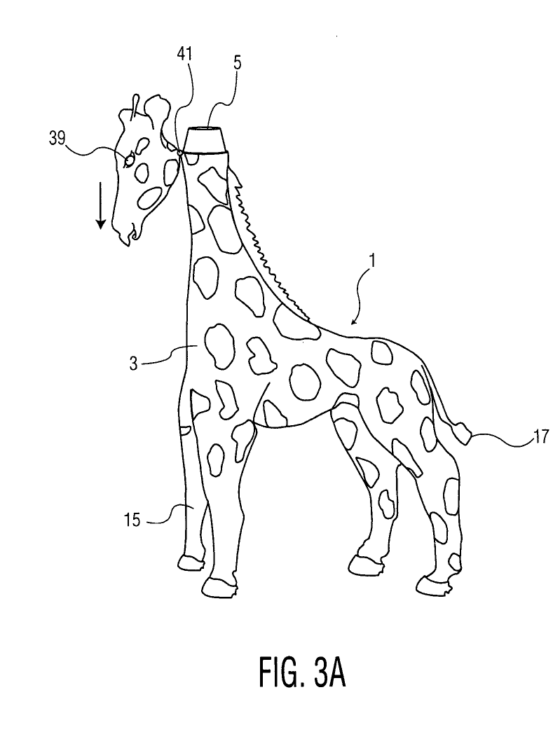

In FIG. 3, the nasal spray dispensing container, 1, is formed in the three-dimensional shape, 3, of a giraffe, with the snout and mouth forming of the insertion region, 7, and the dispensing opening, 5, respectively. The spots, 31, form graphic detail of visual interest the child, and horny protuberances, 13a, near the ears, form fine fingering detail.

FIG. 3A depicts an alternative embodiment of a nasal spray dispensing container according to the present invention. In this embodiment, the dispensing opening, 5, is not the head, or top, of the figurine, but the head of the figurine is removable to expose the dispensing opening, 5. In this particular embodiment, the head is formed as a front-hinged, “flip-top cap, 39, which both moves, but remains attached to the container, by means of hinge, 41. In other embodiments, the head may be completely removable, and re-fastenable, such as a snap-on cap. However, the front-hinged, “flip-top” head not only remains attached, but the front hinge disposes the nostrils of the animal figurine in a nose down position, generally perpendicular to the ground, (note arrow), exactly the position recommended for the administration of nasal spray.

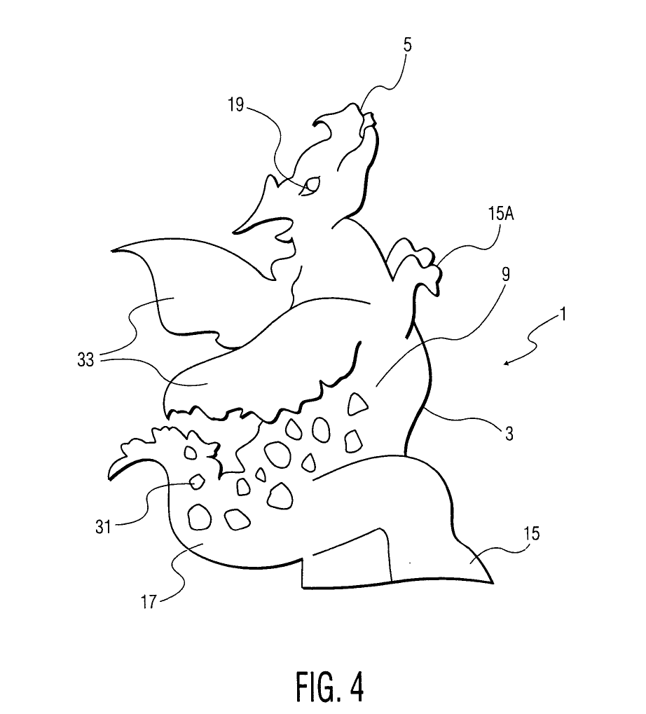

FIG. 4 illustrates a nasal spray dispensing container, 1, formed in the three-dimensional shape, 3, of a dragon. As shown in this embodiment, the front feet, 15a, present fine fingering detail for the child, but only small handles for the user. Graphic detail includes the overall coloring, as well as, e.g., the spots, 31, and eyes, 19. The wings, 33, present not only additional grasping handles for the adult administering the spray, but additional fingering detail for the child.

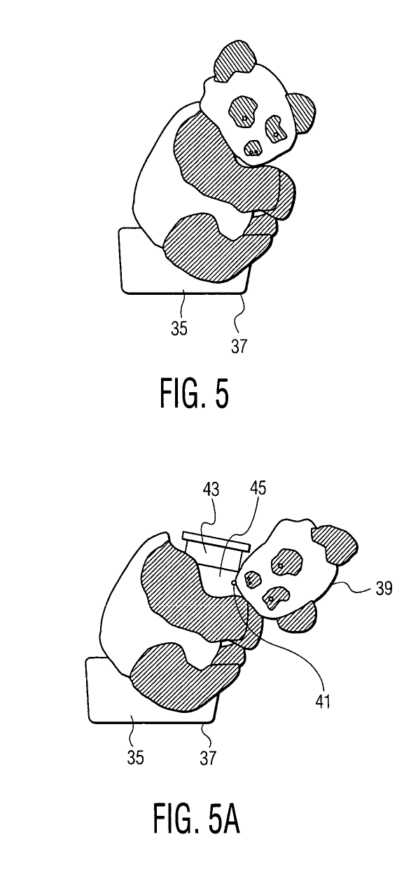

As shown in FIG. 5, a bronchial inhaler may also be formed in the shape of an entertaining figurine, or toy. As shown, the pump action container is disclosed within a three-dimensional figurine of a panda bear. The bear sits atop the breathing, or inhaling, tube, 35. During use, the free end of the breathing/inhaling tube is placed in the mouth, and the inhalant is dispensed by placing the thumb on the remote end of the inhaling tube, 37, and one or more fingers on the bear's head, and pushing down on the head to activate the release of the inhalant mist into the tube, 35.

FIG. 5A is an alternative embodiment of the inhalant mist container of FIG. 5, with a “flip-top” head exposing the plunger, 43, of the inhalant mist canister, 45. As in the embodiment illustrated in FIG. 3A, the hinge, 41, permits movement, but retains the head cap, 39 of the figurine attached to the body of the figurine. In this embodiment, the canister may be removable and replaceable within the figurine container. In this particular embodiment, the person administering the mist pushes the plunger, not the head of the figurine. This embodiment avoids wear and tear of the head of the figurine, and presents a generally flat-topped plunger, which may be easier to press than the head of the figurine.

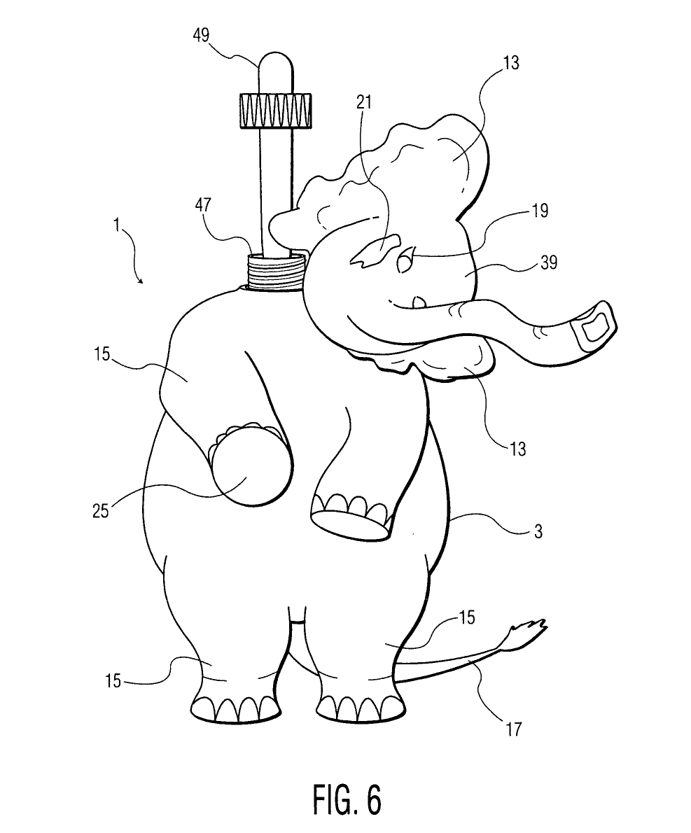

FIG. 6 illustrated another hinged head cap container according to the present invention. In this particular embodiment, the container is adapted to dispense ear drops from dropper, 49. As shown, the head of the figurine is side-hinged, so that when flipped about the hinge, the head rotates to the side, placing the ear of the figurine in an ear-up position, mimicking the position the child must assume to have the drops administered into the ear. Flipping the head about the hinge (not shown), also exposes the dropper, 49, which may be removed from the medicament vessel, in this embodiment, unscrewed from the treaded neck of the bottle, 47. When the child tilts his/her head, and looks the elephant in the eye, they will be ready to receive the ear drops.

Forming the nasal spray dispensing containers, or inhalers, in the shape of an animal, or other entertaining or toy figure, serves a number of purposes. First, the containers of the present invention distract the child from the task at hand, providing a focus of alternative interest. Additionally, forming the nasal spray dispensing container, or inhalers, are intended to provide visual and tactile pleasure, and perhaps instigate play, and cooperation. In addition, the containers provide multiple handles to assist the adult administrator in maintaining a grasp on, and manipulating the position or the container, to accomplish the positioning and coordinated movement required to dispense the spray or mist. In addition, the “flip-top” head may be configured and hinged to mimic the head position of the child during administration of the medicament.

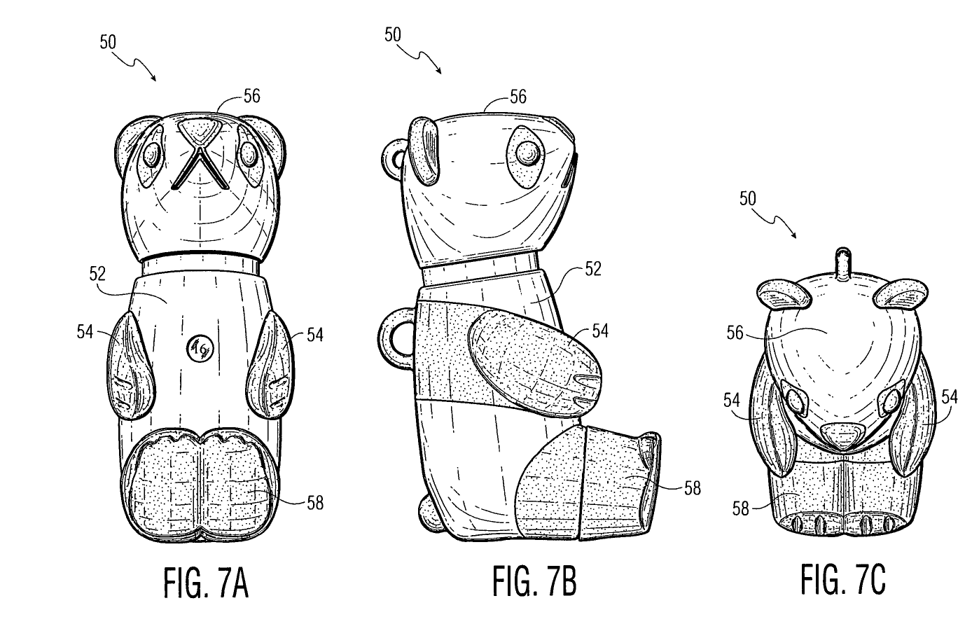

FIGS. 7A, 7B and 7C show the front, side and top views respectively of another asthma medication inhaler in the shape of a panda bear. This inhaler comprises an animal figurine 50 having a torso 52 with arms 54; a head 56 and a pair of hind legs and feet 58. The head 56 is removable to permit insertion of a medication canister; the feet 58 are removable to provide an opening for the inhalant to be dispensed into the user's mouth.

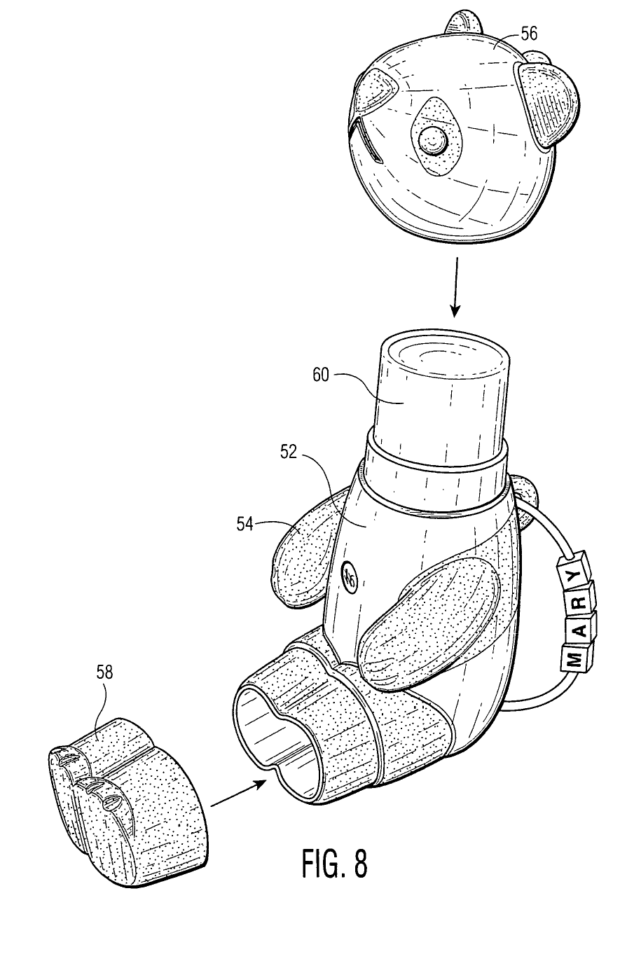

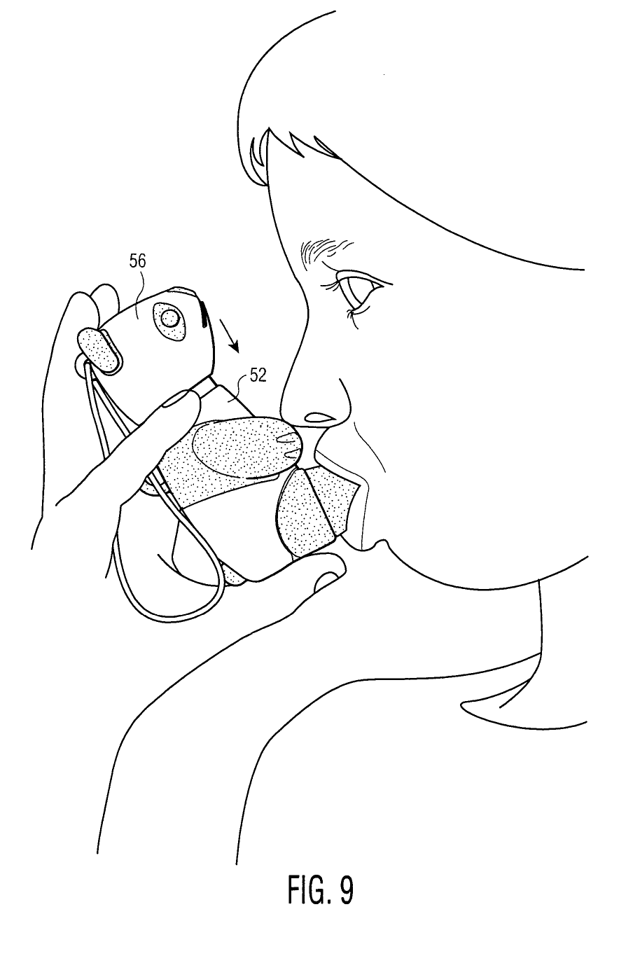

As shown in FIG. 8, the torso 52 is adapted to hold a medication canister 60. The canister releases the medication when the head 56 is depressed by the user in a downward direction, toward the torso 52. FIG. 9 illustrates the proper use of the inhalant dispenser.

As shown in FIGS. 8, 10 and 11, a loop may be provided at the back of the head 56 and/or torso 52 of the dispenser to accommodate and retain a cord or chain which may conveniently hold a tag or the like with the user's name and address and/or may form a necklace that can be passed around the user's neck for convenience in carrying the inhaler.

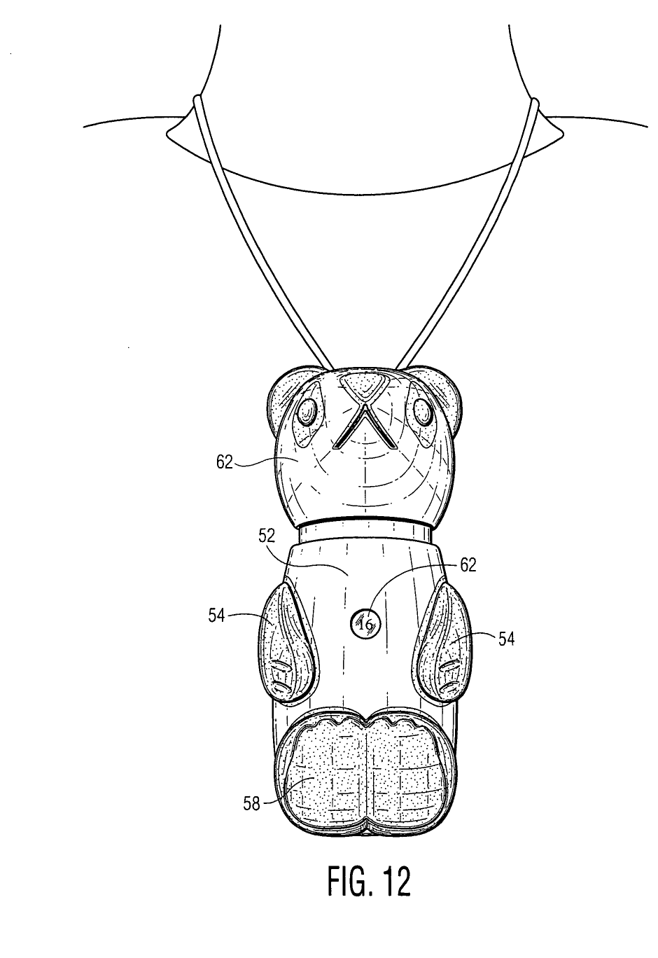

Preferably, the inhalant dispenser incorporates a mechanism for counting the number of puffs of medication dispensed in metered doses from the medication canister. As shown in FIG. 12, the dispenser is provided with a small window opening 62, conveniently in the area of the animal's “belly button”, to display the count (either up count or down count).

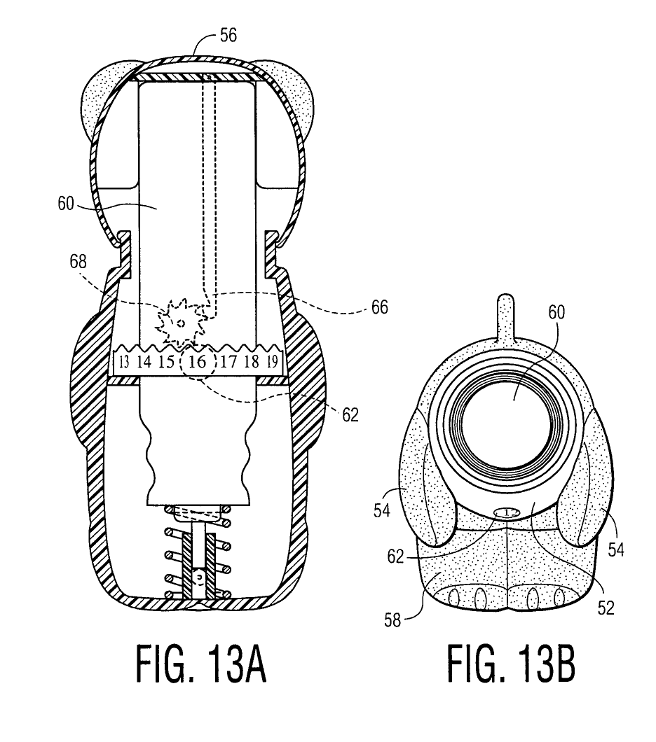

FIGS. 13A and 13B illustrate one embodiment of the counting mechanism according to the invention. As is shown, this mechanism includes a ratchet rod 66 which engages a small pinion gear 68, rotating this gear when the head 56 is depressed. The gear, in turn, increments an elongate strip, in this case a plastic tape with ratchet teeth along one edge, to move the next one of the numbers imprinted on the tape in front of the window opening 62. The tape is arranged in a coil that wraps around the medication canister 60. It is sufficiently long to have imprinted thereon numbers ranging from zero to the maximum number of puffs that the medication canister can dispense; e.g., 200. The lowest group of numbers—for example, 0-25—if the counter counts down, or the highest group of numbers—for example, 175-200—if the counter counts up are preferably in red while the rest are black to warn the user that the canister is approaching its last puff.

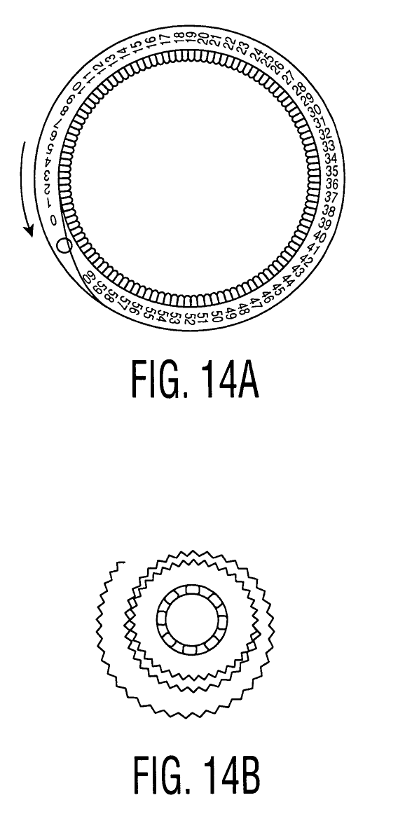

FIGS. 14A and 14B further illustrate the numbered tape which is coiled around the medication canister.

As is conventional with asthma inhalers of this type, the dispensing tube of the medication canister is spring loaded so that the panda head 56 will be biased upwardly, and thus return to its rest position after it has been depressed to dispense a puff. However, one or more additional springs, such as coil springs, may be provided as necessary to support the operation of the counting mechanism.

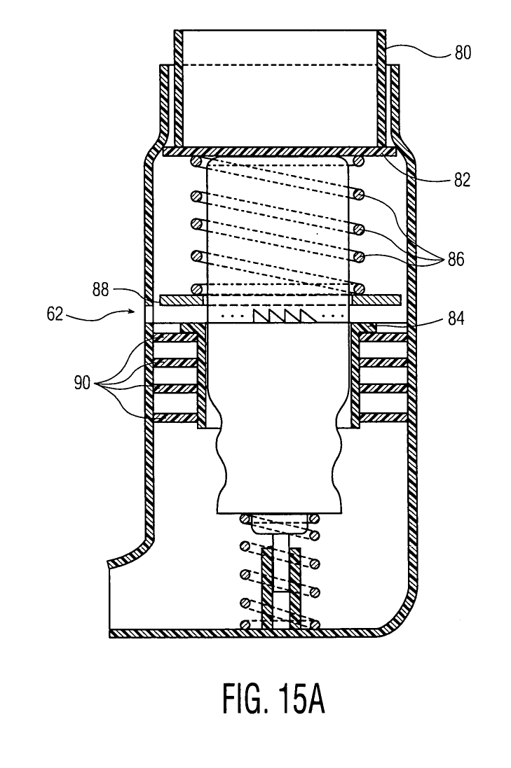

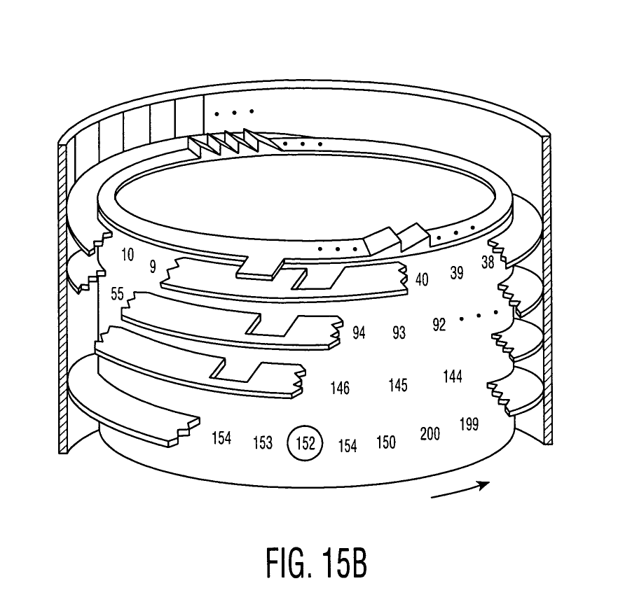

FIGS. 15A and 15B show an alternative embodiment of the counting mechanism which employs a cylinder 80 with numbers imprinted thereon in place of an elongate strip. This cylinder is rotatable in increments upon each depression of the panda head 56 by a mechanism similar to that used in “click type” ball point pens to successively protrude and retract and the tip of the ink cartridge when the actuation button, at the top of the pen, is repeatedly depressed. In this case, the cylinder is caused to rotate about its axis 82 to successively increment numbers in front of the window opening 60 of the inhaler. The numbers 0-200 are arranged in four rows around the cylinder. As is best seen in FIG. 15B, the cylinder 80 is arranged to not only rotate about its axis 82 but also move axially in increments to align successive ones of the rows of numbers with the window opening 62.

As indicated in 15A, the cylinder 80 is continuously biased in the downward direction by a coil spring 86 and washer 88. However, the cylinder is prevented from moving downward by a tab 84 which rests on one of four circular shelves 90 integrated onto the inside surface of the panda bear torso 52. After the cylinder makes one complete revolution, the tab 84 drops through an opening in the retaining shelf 90 and lands on the next successive shelf 90, incrementing the cylinder axially by the distance of one number row. Details of this mechanism are shown in FIG. 15B. The transverse movement of the cylinder 80 will occur three times as the cylinder traverses the first three shelves until the tab 84 finally rests on the lower-most shelf 90 and the top-most row of numbers is aligned with the window opening 62. In this way, 200 numbers may be displayed from a single cylinder which holds for rows of numbers with 50 numbers in each row.

There has thus been shown and described a novel container for a nasal spray, or an inhaler, which fulfills all the objects and advantages sought therefore. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

1. A nasal spray dispensing container comprising an entertaining figurine, containing a vessel for nasal spray, a dispensing opening, and means for dispensing the nasal spray through the dispensing opening.

2. A squeeze bottle nasal spray dispensing container wherein the container is formed in the shape of an entertaining figurine.

3. The nasal spray dispensing container of claim 2, further comprising additional means for grasping the container, comprising the appendages of the figurine.

4. The nasal spray dispensing container of claim 2, further comprising fingering details of the figurine.

5. The nasal spray dispensing container of claim 2, further comprising graphic details of the figurine.

6. The nasal spray dispensing container of claim 1, wherein the figurine comprises a three dimensional unicorn with appendages selected from the group consisting the head, legs, feet, tail and mane, which provide handles for grasping the container.

7. The nasal spray dispensing container of claim 1, wherein the figurine comprises a three dimensional elephant having appendages selected from the group consisting the head, ears, legs, feet, tail, truck and tusks, which provide handles for grasping the container, and wherein the dispensing opening is disposed in the trunk.

8. The nasal spray dispensing container of claim 1, wherein the figurine comprises a three dimensional giraffe having appendages selected from the group consisting the head, neck, horny protuberances near the ears, legs, feet, and tail, which provide handles for grasping the container, and wherein the dispensing opening is disposed in the head.

9. The nasal spray dispensing container of claim 1, wherein the figurine comprises a three dimensional dragon having appendages selected from the group consisting the head, wings, legs, feet, and tail, which provide handles for grasping the container, wherein the dispensing opening is disposed in the head.

10. A pump action nasal spray dispensing container comprising the shape of an entertaining figurine, containing a vessel for nasal spray, a dispensing opening, and means for dispensing the nasal spray through the dispensing opening.

11. The nasal spray dispensing container of claim 10, further comprising additional means for grasping the container, comprising the appendages of the figurine.

12. The nasal spray dispensing container of claim 10, further comprising fingering details of the figurine.

13. The nasal spray dispensing container of claim 10, further comprising graphic details of the figurine.

14. A container for an inhaler, said container comprising an entertaining figurine.

15. A pump action inhalant mist dispenser comprising the three dimensional shape of an entertaining figurine, containing a canister for the inhalant, an inhaling tube, and means for dispensing a mist of the inhalant.

16. The pump action inhalant dispenser of claim 15, further comprising additional means for grasping the container, comprising the appendages of the figurine.

17. The pump action inhalant dispenser of claim 15, further comprising fingering details of the figurine.

18. The pump action inhalant dispenser of claim 15, further comprising graphic details of the figurine.

19. An inhalant dispenser comprising an animal figurine having a torso for holding a canister of inhalant and a head and feet attached to the torso.

20. The inhalant dispenser of claim 19, wherein the animal figurine is a panda bear.

21. The inhalant dispenser of claim 19, wherein the head is removable for insertion of a canister of inhalant, and the feet are removable to provide an opening for the inhalant to enter the user's mouth.

22. The inhalant dispenser of claim 21, wherein a puff of inhalant is released from the canister upon pressing the head downward toward the torso.

23. The inhalant dispenser of claim 22, further comprising a counter mechanism for counting the number of times that the head is pressed downward, thereby counting the number of puffs produced by the canister of inhalant.

24. The inhalant dispenser of claim 23, wherein the counter mechanism includes a rack and pinion gear for changing downward linear motion of the head into rotary motion.

25. The inhalant dispenser of claim 24, wherein the counter mechanism further includes an elongate strip arranged within the torso and having a succession of numbers imprinted thereon for display of the number of puffs as it moves in front of a window opening in the torso.

26. The inhalant dispenser of claim 25, wherein the counter mechanism further includes a ratchet which permits the elongate strip to move in one direction only.

27. The inhalant dispenser of claim 22, further comprising a spring for biasing the head in the upward direction.

28. The inhalant dispenser of claim 22, wherein the counter mechanism includes a cylinder, arranged within the torso and having a succession of numbers imprinted thereon for display of the number of puffs.

29. The inhalant dispenser of claim 28, wherein the numbers are imprinted in a plurality of rows on said cylinder.

30. The inhalant dispenser of claim 29, wherein the counter mechanism further includes means for shifting the cylinder in the direction of its axis to align one of the rows of numbers with a window opening in the torso.

31. The inhalant dispenser of claim 28, wherein the counter mechanism further includes means for rotationally incrementing the cylinder about its axis to align one of the numbers with a window opening in the torso.

32. The inhalant dispenser of claim 31, wherein the incrementing means includes a ratchet.

33. A nasal spray container as in claim 2, wherein the figurine has a head, separable from the body of the figurine, said head covering the dispensing opening.

34. A nasal spray dispensing container as in claim 33, further comprising a hinge, said head being attached the body by the hinge.

35. A nasal spray container as in claim 34, wherein the figurine depicts an animal with nostrils, and the hinge is attached to the front of the head of the figurine, such that when the head is moved about the hinge, exposing the dispensing opening, the nostrils of the figurine are perpendicular to the ground.

36. A container for ear drops, said container comprising an entertaining figurine containing a vessel for the ear drops, a removable but replaceable vessel cap, and a dropper.

37. A container for ear drops as in claim 36, wherein the figurine comprises an animal having appendages selected from the group consisting of the head, legs, feet, neck and tail, which provide handles for grasping and holding the container.

38. A container for ear drops as in claim 36, wherein the figurine has a head, separable from the body of the figurine, said head covering the cap and dropper.

39. A container for ear drops as in claim 36, further comprising a hinge, said head being attached the body by the hinge.

40. A container for ear drops as in claim 39, wherein hinge is placed at the side of the head, such that when the head is rotated about the hinge, exposing the cap and stopper, the head of the figurine is generally perpendicular to the body.

41. The container for ear drops as in claim 39, wherein the figurine has ears, and the ears face up when the head of the figurine is rotated about the hinge.
